(19) United States
(12) Patent Application Publication
(10) Pub. No.: US 2016/0194273 A1
(43) Pub. Date: Jul. 7, 2016

(54) PROCESS FOR THE ETHERIFICATION OF AMINO ALCOHOLS WITH METAL ALCOHOLATES

(71) Applicant: BASF SE, Ludwigshafen am Rhein (DE)

(72) Inventors: Melanie WEINGARTEN, Ratzeburg (DE); Wolfgang SIEGEL, Limburgerhof (DE)

(73) Assignee: BASF SE, Ludwigshafen am Rhein (DE)

(21) Appl. No.: 14/987,960

(22) Filed: Jan. 5, 2016

(30) Foreign Application Priority Data
Jan. 6, 2015 (EP) 15150183

(51) Int. Cl. C07C 213/06

(52) U.S. Cl. CPC C07C 213/06

(57) ABSTRACT

Process for the etherification of amino alcohols with metal alcoholates

A process for the preparation of the ether of formula I

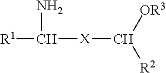

where R₁ and R₂ independently from one another are hydrogen or an alkyl group with 1 to 10 C atoms, R₃ is an alkyl group with 1 to 10 carbon atoms and

X is a bond or a hydrocarbon group with 1 to 10 carbon atoms

comprising

a) deprotonating the amino alcohol of formula II

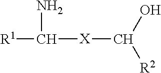

where R₁, R₂ and X have the meaning above

with a metal alcoholate as deprotonating agent to give the anion of formula III

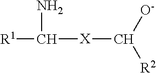

where R₁, R₂ and X have the meaning above

and

b) alkylation of the anion obtained in step a) with an alkylation agent to give the ether of formula I,

wherein the deprotonating agent in step a) is used in equimolar or less than equimolar amounts compared to the amino alcohol and

the alkylation agent in step b) is used in equimolar or less than equimolar amounts compared to the anion of formula III.

The present invention relates to a process for the preparation of the ether of formula I

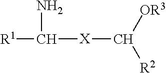

where R₁ and R₂ independently from one another are hydrogen or an alkyl group with 1 to 10 C atoms, R₃ is an alkyl group with 1 to 10 carbon atoms and
X is a bond or a hydrocarbon group with 1 to 10 carbon atoms
comprising
a) deprotonating the amino alcohol of formula II

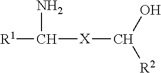

where R₁, R₂ and X have the meaning above

with a metal alcoholat as deprotonating agent to give the anion of formula III

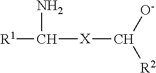

where R₁, R₂ and X have the meaning above

and

b) alkylation of the anion obtained in step a) with an alkylation agent to give the ether of formula I,

wherein the deprotonating agent in step a) is used in equimolar or less than equimolar amounts compared to the amino alcohol and

the alkylation agent in step b) is used in equimolar or less than equimolar amounts compared to the anion of formula III.

Ethers of formula I are chemical intermediates which are, for example, used for the synthesis of pharmaceuticals, plant protecting agents as herbicides, insecticides or fungicides.
Compounds of formula I may be obtained by etherification of amino alcohols. As such amino alcohols have two functional groups (a primary amino group and a hydroxy group) a selective etherification of the hydroxy group becomes problematic. Mono- or di-alkylation of the nitrogen atom will occur sometimes even preferentially. In particular the by-product with a mono-alkylated nitrogen has to be avoided as the boiling point of such by-product is quite similar to the boiling point of the desired ether of formula I. Hence any separation of such by-product by distillation becomes difficult.
In addition, it is often required that the compound of formula I is a defined stereo isomer. Hence an amino alcohol with a defined stereo isomerism is selected as starting material, for example a pure (S) or (R) isomer. Any isomerization during the preparation of the ether has to be avoided and the ether obtained should finally have the same stereo isomerism as the amino alcohol.
DE-A 103 44 447 discloses a process for the etherification of amino alcohols having an unsubstituted amino group and a hydroxyl group. The process described is a two-step process. In a first step an alkali alcoholate is used to prepare the alcoholate of the amino alcohol. In a second step the alcoholate of the amino alcohol is alkylated with an alkylating agent to finally form the corresponding ether. In the example 0.67 mol of the the alkylating agent (chlormethane) are used on 0.5 mol of the alcoholate anion; the alcoholate anion being obtained by reacting 0.66 mol of L-phenylglycinol with 0.5 mol sodium methylate.
It is the object of the present invention to improve the process for the preparation of ethers of formula I. The improved process should be very effective and easy to perform. The yield of amino alcohol ethers should be high and any by-products, specifically by-products with substitution at the nitrogen should be avoided. The overall selectivity of the ethers and the retention of the stereo chemistry should be as high as possible.
Accordingly, a process as defined above has been found.
To the ether for formula I
This claimed process is a process for the preparation of ether of formula I

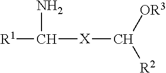

where R₁ and R₂ independently from one another are hydrogen or an alkyl group with 1 to 10 C atoms, R₃ is an alkyl group with 1 to 10 carbon atoms and X is a bond or a hydrocarbon group with 1 to 10 carbon atoms.
Preferably R₁ and R₂ independently from each other are hydrogen or an alkyl group with 1 to 4 C atoms.
Preferably R₃ is an alkyl group with 1 to 4 C atoms.
Preferably X is a bond or an alkylene group with 1 to 10 carbon atoms. In a most preferred embodiment X is a bond.
In a particularly preferred embodiment the compound of formula I is a compound wherein R₁ is hydrogen or an alkyl group with 1 to 4 C atoms, R₂ is hydrogen, R₃ is an alkyl group with 1 to 4 C atoms and X is a bond or an alkylene group with 1 to 10 carbon atoms.
In a most preferred embodiment the compound of formula I is a compound wherein R₁ is a methyl group, R₂ is hydrogen, R₃ is a methyl group and X is a bond. This compound is known as 1-methoxy-2-propylamin.
Preferably the compound of formula I has a defined stereo isomerism. In particular it may be a (R) or (S) enantiomer or a defined mixture thereof, the chiral carbon atom being the carbon atom to which the primary amino group is bonded. Particularly preferred is a pure (R) or (S) enantiomer.
In a most preferred embodiment the compound of formula I is (S)-1-methoxy-2-propylamin.
To process step a)
In process step a) an amino alcohol of formula II

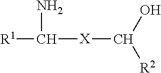

is deprotonated with a metal alcoholat as deprotonating agent to give the anion of formula III

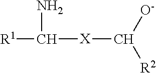

In both formulas II and III R₁, R₂, R₃ and X have the meanings and preferred meanings above.
In the most preferred embodiment the amino alcohol of formula II is alaninol, in particular pure (S)-alaninol (R₁=Methyl, R₂=H, X=bond)
Preferred metal alcoholates are alcoholates of metals of group I to III of the periodic system, in particular alkali metal alcoholates or earth alkali metal alcoholates.
Most preferred are alkali metal alcoholates.
Preferred metal alcoholates are those of hydrocarbons with one hydroxyl group, in particular a metal C1- to C10-alkylate, respectively a metal C1- to C4-alkylate.
Most preferred deprotonating agents are for example lithium C1- to C4-alkylates, sodium C1- to C4-alkylates or potassium C1- to C4-alkylates, in particular sodium methylate, sodium ethylate, potassium methylate or potassium ethylate. Particularly preferred are sodium C1- to C4-alkylates, namely sodium methylate.
Process step a) may be performed in presence of a solvent or in the absence of a solvent.
In the absence of a solvent the amino alcohol (formula II) and/or the ether obtained (formula I) would have partially the function of a solvent.
In a preferred embodiment step a) is performed in the presence of a solvent.
Suitable solvents are, for example, aliphatic solvents, for example C5- to C16-alkanes like hexane, cyclohexane, heptane, or aromatic solvents. An aromatic solvent is a solvent with at least one aromatic ring system, an aromatic solvent may contain other organic groups than the aromatic ring system as, for example, alkyl or alkoxy groups as subsituents to the aromatic ring system.
Toluene, xylene, for example ortho-xylene, meta-xylene , para-xylene or mixtures thereof, anisol, ether including cyclic ethers, e.g. dioxane, tetrahydrofurane, or ethyleneoxide ethers, in particular glymes like 1,2-dimethoxyethane (monoglyme), diethyleneglycoldimethylether (diglyme), 1,2-diethoxyethane (ethylglyme), diethoxy-diethylene glycol (ethyl diglyme), diethylene glycol dibutylether (butyl diglyme) et al. or polyethers like poly(ethylene glycol) dimethylether et al.
Preferred solvents are hydrophobic solvents, in particular aliphatic or aromatic hydrocarbons or ethers, as for example C7- to C15 alkanes, alkylaromatic solvents or ethyleneoxide ethers.
More preferred are aromatic solvents, in particular aromatic hydrocarbons like toluene or xylene.
Preferably a mixture of the amino alcohol and a solvent is used in process step a).
Preferably, the amino alcohol, respectively the mixture, should be free of water and other protic solvents.
The metal alcoholate may be used in solid or liquid form. The liquid form may be the molten metal alcoholate or a solution of the metal alcoholate. Preferably the metal alcoholate is used in solid form or in form of a solution. Most preferred is the use of the metal alcoholate in solution, in particular as a solution in an aliphatic alcohol, preferably a solution in methanol.
The metal may be added to the amino alcohol, the solvent or the mixture thereof.
Process step a) is preferably performed at a temperature of at least the melting temperature of the metal alcoholate used as deprotonating agent.
The temperature may be, for example, at minimum 40° C. The temperature may be, for example, at maximum 200° C., respectively 160° C. or 140° C. A very suitable range of temperatures is, for example 40 to 120° C.
Process step a) may be performed at normal, at reduced or at increased pressure. Usually process step a) will be performed at a pressure of 0.4 to 3 bars, in particular at 0.8 to 1.5 bar and preferably simply at normal pressure (1 bar).
The metal alcoholate in step a) is used in equimolar or less than equimolar amounts compared to the amino alcohol.
Preferably, metal alcoholate in step a) is used in less than equimolar amounts compared to the amino alcohol.
The ratio of equivalents of the amino alcohol to the metal alcoholate is preferably from 1:0.99 to 1:0.6, in particular from 1:0.98 to 1:0.90.
To process step b)
In process step b) the anion of formula III obtained in step a) is alkylated. The reaction under step b) is preferably started when all metal alcoholate in process step a) is consumed.
Suitable alkylating agents are well known. Usual alkylating agents correspond to the general formula V

(Alkyl)ₘ—Z,

wherein Alkyl is an alkyl group, preferably an alkyl group with 1 to 4 carbon atoms, most preferred a methyl group, m may be 1, 2 or 3 and Z is a one, two or three valent inorganic or organic, corresponding the actual meaning of n.
In particular n is 1 or 2.
In particular Z is a halogen, for example chloride, or an organic or inorganic ester group.
As examples alkyl chloride, alkyl mesylate, alkyl tosylate, dialkyl sulfate or dialkyl carbonate, trialkyl phosphate may be named.
As R₃ preferably is a methyl group, the preferred reaction is a methylating reaction using, for example, methyl chloride, dimethyl sulfate or dimethyl carbonate as methylating agent.
In one preferred embodiment the alkylating agent, respectively methylating agent, is used as a gas. The boiling point of methyl chloride is −23.8° C. at 1 bar.
In a preferred embodiment of step a) a solvent has been used. This solvent is preferably not removed in or before step b). Hence both, process step a) and b) are preferably performed in presence of the same solvent, in particular aromatic hydrocarbons like toluene or xylene.
In step b) the alkylation agent is used in equimolar or less than equimolar amounts compared to the anion of formula III.
Preferably, the alkylation agent is used in less than equimolar amounts compared to the anion of formula III.
The ratio of equivalents of the anion of formula III to the alkylation agent is preferably from 1:0.99 to 1:0.6, in particular from 1:0.98 to 1:0.90.
Process step b) may be performed at elevated temperature.
The temperature may be, for example, at minimum 30° C., respectively 60° C. The temperature may be at maximum 200° C., respectively 160° C. or 140° C., in particular at maximum 120° C. A very suitable range of temperatures is, for example 60 to 140° C., respectively 60 to 120° C.
Process step b) may be performed at normal, at reduced or at increased pressure. Process step b) may be for example performed at a pressure of 0.4 to 3 bars, in particular at 0.8 to 1.5 bar and preferably simply at normal pressure (1 bar).
The following reaction scheme shows process steps a) and b) for alaninol, sodium methylate and methyl chloride:

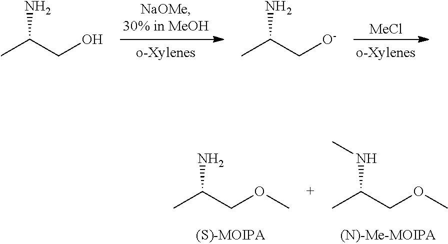

Process step b) usually results in a suspension comprising the ether of formula I, salts that have been formed from the metal cation and the remaining group of the alkylating agent, for example sodium chloride, organic solvent that has been used and further by-products.
The suspension usually comprises solid salts. In order to withdraw such salts from said suspension, the salts may be filtered off. Such filtration would usually be done before any distillation.
The ether of formula I may be distilled from such suspension or solution and thereafter purified, for example by further distillation.
Alternatively the salts may be removed after the distillation of the ether by adding an amount of water which is sufficient to solve any salts. The obtained aqueous salt solution and the solvent form distinct phases and can be separated easily.
By-products that may have been formed are compounds with substitution at the nitrogen atoms, in particular compounds wherein the nitrogen atom is alkylated as well, either once (giving a secondary amino group) or even twice (giving a tertiary amino group).
It is advantage of the claimed process that such by-products are not or at least hardly formed.
In particular the molar ratio of the ether of formula I to the by-product which is N-mono alkylated as well is usually more than 8:1, preferably more than 10:1.
The selectivity of the ether of formula I is usually more than 80%, in particular more than 90%, such selectivity being the ratio of the equivalents of the ether of formula I to the sum of the equivalents of all and any alkylated compounds ×100%.
It is a further advantage of the claimed process that the stereo isomerism of the amino alcohol is kept. Usually at least 90%, typically at least 95%, respectively at least 98% of the ether of formula I obtained by the process has the same stereoisomerism as the amino alcohol. Hence starting with (S)-alaninol will give by methylating at least 90%, typically at least 95%, respectively at least 98% of the 1-methoxy-2-propylamine obtained is (S)-1-methoxy-2propylamin.
Furthermore the process claimed is a very effective and efficient process. The process allows high yields of ether.

### EXAMPLES

MOIPA shall be any 1-methoxy-2-propylamin
(S)-MOIPA shall be the stereo isomer (S)-1-methoxy-2-propylamin.
N-Me-MOIPA shall be (S)-1-methoxy-2-propylamin with an additional methylation at the nitrogen atom (IUPAC-name: (2S)-1methoxy-N-methyl-propan-2-amine).
Gas chromatography (Hewlett Packard 6890 N with FID detector; column: 25 m Hydrodex-yTBDAc, inner diameter 0.25 mm, outer diameter 0.40 mm, film diameter 0.25 pm, oven program: temperature start: 60° C., hold: 0 min, in 5 K /min steps to temperature end: 220° C., hold: 1 min; column: 30 m Optima 5 MS, inner diameter 0.25 mm, outer diameter 0.45 mm, film diameter 1.0 pm, oven program: temperature start: 60° C., hold: 5 min, in 15 K /min steps to temperature end: 280° C., hold: 1 min.) was used to determine the yield and selectivity. The yield and selectivity were calculated from the area of the peaks of the gas chromatogram using calibration methods.

### Comparison Example (Excess of Alkylating Agent)

80 g of L-alaninol (1.06 mol) and 485 g of o-xylenes (4.57 mol) were added to a 0.75 liter reactor (with Rushton type impeller and pitched-blade impeller) and brought to 250 mbar and 40-50° C. at 400 rpm. During 1.5 h 172.4 g of sodium methylate (30 wt% in methanol, 0.96 mol) were added dropwise at temperature (in) =44-60° C. and temperature (head) =21-33° C. while methanol was distilled off. Methanol and o-xylene removal is continued until the boiling point of o-xylene of is reached. Afterwards, 47 g of o-xylene are added towards the suspension which is the amount of o-xylene that was removed via distillation.
The suspension was transferred at 55° C. to a 0.75 liter glass pressure reactor (with Rushton type impeller and pitched-blade impeller) and at temperature (in) =59-78° C. and 800 rpm 54.8 g of methyl chloride (1.09 mol) was added under pressure in four portions (0.2-0.8 bar) over 30 min. After methyl chloride addition was finished the reaction mixture was stirred for further 2 hours at a temperature of 70° C. and subsequently brought to room temperature.
The composition of the product mixture obtained was analyzed.
More than 99.9% of the MOIPA obtained were (S)-MOIPA.
The yield of (S)-MOIPA was 40% (based on sodium methylate).
The ratio of (S)-MOIPA to the byproduct N-Me-MOIPA was 7:1 (ratio of the corresponding areas of the peaks of the gas chromatogram).
The selectivity of (S)-MOIPA was 80%.

### Example 1

### 1 Eq L-Alaninol: 0.95 Eq Sodium Methylate: 0.93 Eq Methylchloride

80 g of L-alaninol (1.06 mol) and 485 g of o-xylenes (4.57 mol) were added to a 0.75 liter reactor (with Rushton type impeller and pitched-blade impeller) and brought to 250 mbar and 50-60° C. at 400 rpm. During 1 h 15 min 181.9 g of sodium methylate (30 wt % in methanol, 1.01 mol) were added dropwise at a temperature of 53 to 63° C. while methanol was distilled off. Methanol and o-xylene removal is continued until the boiling point of o-xylene of is reached. Afterwards, 53 g of o-xylene are added towards the suspension which is the amount of o-xylene that was removed via distillation.
The suspension was transferred at 60° C. to a 0.75 liter glass pressure reactor (with Rushton type impeller and pitched-blade impeller) and at temperature of 59 to 66° C. and 800 rpm 50 g of methyl chloride (0.99 mol) was added under pressure in five portions (0.1-0.4 bar) over 60 min. After methyl chloride addition was finished the reaction mixture was stirred for further 2 hours at temperature of 70° C. and subsequently brought to room temperature.
The composition of the product mixture obtained was analyzed.
More than 99.9% of the MOIPA obtained were (S)-MOIPA.
The yield of (S)-MOIPA was 49% (based on methyl chloride).
The ratio of (S)-MOIPA to the byproduct N-Me-MOIPA was 9:1 (ratio of the corresponding areas of the peaks of the gas chromatogram).
The selectivity of (S)-MOIPA was 85%.

### Example 2

### 1 Eq L-Alaninol: 0.93 Eq Sodium Methylate: 0.85 Eq Methylchloride

80 g of L-alaninol (1.06 mol) and 485 g of o-xylenes (4.57 mol) were added to a 0.75 liter reactor (with Rushton type impeller and pitched-blade impeller) and brought to 250 mbar and 50-60° C. at 400 rpm. During 35 min 178.1 g of sodium methylate (30 wt% in methanol, 0.99 mol) were added dropwise at temperature of 53 to 58° C. while methanol was distilled off. Methanol and o-xylene removal is continued until the boiling point of o-xylene of is reached. Afterwards, 133 g of o-xylene are added towards the suspension which is the amount of o-xylene that was removed via distillation.
The suspension was transferred at 60° C. to a 0.75 liter glass pressure reactor (with Rushton type impeller and pitched-blade impeller) and at temperature of 63 to 79° C. and 800 rpm 45.6 g of methyl chloride (0.9 mol) was added under pressure in five portions (0.3-0.8 bar) over 60 min. After methyl chloride addition was finished the reaction mixture was stirred for further 2 hours at temperature of 70° C. and subsequently brought to room temperature.
The composition of the product mixture obtained was analyzed.
More than 99.9% of the MOIPA obtained were (S)-MOIPA.
The yield of (S)-MOIPA was 50% (based on methyl chloride).
The ratio of (S)-MOIPA to the byproduct N-Me-MOIPA was 10:1 (ratio of the corresponding areas of the peaks of the gas chromatogram).
The selectivity of (S)-MOIPA was 84%.

### Example 3:

### 1 Eq L-Alaninol: 0.95 Eq Sodium Methylate: 0.93 Eq Methylchloride

327 g of diethyleneglycoldibutylether (1.5 mol) were added to a 0.75 L reactor (with impeller stirrer and 4 baffles) and brought to ˜125° C. 98.03 g of sodium methylate (1.76 mol) were added over 2 min at ˜125° C. (450 rpm). The reaction mixture was stirred for further 15 min. A white good stirrable suspension was obtained.
140 g of L-alaninol (1.85 mol) were added dropwise under control of temperature during 1 h and 30 min at a temperature of 126 to 128° C. (450 rpm). A white very thick suspension was obtained. The reaction mixture was stirred further for one hourh at 450-600 rpm and a temperature of 108 to 115° C. A white thin suspension was obtained afterwards.
38.4 g of Methanol (96.3 GC-a%) were removed by distillation at 600-100 mbar and a temperature of 125-127° C. The reaction mixture was further stripped with argon for 4 hours. However, no further distillate was obtained. The reaction mixture was brought to 100° C. A thick, but good stirrable yellowish suspension was obtained.
87 g of methyl chloride (1.72 mol) were inserted during 16 h 10 min at a temperature of 103-106° C. (300 - 500 rpm) into the suspension. A thin, slightly yellow suspension was obtained. The reaction mixture was filtered (filter resistance of 2.8*1013 mPas/m²).
The composition of the product mixture obtained was analyzed.
More than 99.9% of the MOIPA obtained were (S)-MOIPA.
The yield of (S)-MOIPA was 53% (based on methyl chloride).
The ratio of (S)-MOIPA to the byproduct N-Me-MOIPA was 24:1 (ratio of the corresponding areas of the peaks of the gas chromatogram).
The selectivity of (S)-MOIPA was 93%.

### Example 4

### Dimethyl Sulfate as Alkylating Agent, 1 Eq L-Alaninol: 1Eq Sodium Methylate: 1 Eq Dimethyl Sulfate

33.85 g (L)-alaninol (0.45 mol) in 169 g isomeric mixture of xylenes (1.59 mol) are brought to 110° C. under a vacuum of 600 mbar. At 90 to 100° C. 80.83 g sodium methylate (30 wt. % in methanol, 0.45 mol) is added dropwise over 60 min. During addition methanol is removed by distillation (reflux ratio 5:1). After addition is finished distillation is continued until the transition temperature of xylenes 118° C. is reached. Meanwhile 70 mL of an isomeric mixture of xylenes is added to keep the amount of xylenes roughly constant.
The opaque yellow reaction mixture is stirred at 72-87° C. 57.19 g dimethylsulfate (0.45 mol) is dissolved in 45 mL of an isomeric mixture of xylenes and added dropwise over 60 min and in parallel the product (S)-MOIPA is removed at 200 mbar via distillation (reflux ratio of 5:1). After addition is finished distillation is continued until temperature (head) =87° C. is reached. 34.8 g of (S)-MOIPA as a colourless clear liquid is obtained.
The composition of the product mixture obtained was analyzed.
More than 99.6% of the MOIPA obtained were (S)-MOIPA.
The yield of (S)-MOIPA was 26%.
The ratio of (S)-MOIPA to the byproduct N-Me-MOIPA was 13:1 (ratio of the corresponding areas of the peaks of the gas chromatogram).
The selectivity of (S)-MOIPA was 92%.

1. A process for the preparation of the ether of formula I 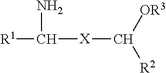
where R₁ and R₂ independently from one another are hydrogen or an alkyl group with 1 to 10 C atoms, R₃ is an alkyl group with 1 to 10 carbon atoms and
X is a bond or a hydrocarbon group with 1 to 10 carbon atoms
comprising
a) deprotonating the amino alcohol of formula II 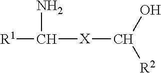
where R₁, R₂ and X have the meaning above
with a metal alcoholate as deprotonating agent to give the anion of formula III 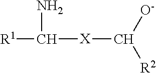
where R₁, R₂ and X have the meaning above
and
b) alkylation of the anion obtained in step a) with an alkylation agent to give the ether of formula I,
wherein the deprotonating agent in step a) is used in equimolar or less than equimolar amounts compared to the amino alcohol and
the alkylation agent in step b) is used in equimolar or less than equimolar amounts compared to the anion of formula III.

2. A process according to claim 1, wherein the deprotonating agent in step a) is used in less than equimolar amounts compared to the amino alcohol and the alkylation agent in step b) is used in less than equimolar amounts compared to the anion of formula III.

3. A process according to claim 1, wherein R₁ is hydrogen or an alkyl group with 1 to 4 C atoms, R₂ is hydrogen, R₃ is an alkyl group with 1 to 4 C atoms and
X is a bond or an alkylene group with 1 to 10 carbon atoms.

4. A process according to claim 1, wherein R₁ is a methyl group, R₂ is hydrogen, R₃ is a methyl group and X is a bond.

5. A process according to claim 1, wherein the compound of formula II is a pure (R) or (S) enantiomer.

6. A process according to claim 1, wherein the metal alcoholate used as deprotonating agent is an alkali or earth alkali metal alcoholate.

7. A process according to claim 1, wherein the metal alcoholate used as deprotonating agent is sodium methylate.

8. A process according to claim 1, wherein the alkylation agent is selected from alkyl chloride, dialkyl sulfate or dialkyl carbonate.

9. A process according to claim 1, wherein process steps a) and b) are performed in presence of a solvent.

10. A process according to claim 1, wherein process steps a) and b) are performed in presence of an aromatic solvent.
